(19) United States
(12) Patent Application Publication
(10) Pub. No.: US 2006/0249162 A1
(43) Pub. Date: Nov. 9, 2006

(54) Condom facilitated anatomical placement of a topical menthol/arginine preparation for optimum stimulation of a female

(76) Inventors: Ronald J. Thompson, Fort Thomas, KY (US); Justin R. Thompson, Cincinnati, OH (US); James M. Thompson, Cincinnati, OH (US); Donald N. Halgren, Manchester, MA (US)

Correspondence Address: Donald N. Halgren, 35 Central St., Manchester, MA, 01944, US

(21) Appl. No.: 11/123,595

(22) Filed: May 7, 2005

(51) Int. Cl. A61F 6/04

(52) U.S. Cl. 128/844

(57) ABSTRACT

A method of improving the use of a condom for delivery of a stimulant or lubricant treatment product to a female. The method comprises the steps of forming a condom on a mold, the condom having a closed distal end and an open proximal end; and applying the treatment product on an arcuate band portion of the proximal end of said condom subsequent to the forming of the condom on the mold.

### BACKGROUND OF THE INVENTION

1. Field of the Invention

This invention relates to condoms and more particularly to condom manufacture wherein a female lubricant and stimulant is arranged thereon.

2. Prior Art

A 1954 U.S. Army produced training video, “The Proper use of a Condom,” instructs that a condom be used by placing “the condom over the end of the penis, and unroll the condom until you run out of one or the other.” All condoms are packaged rolled up and are meant to be fitted onto an erect penis by a method of unrolling. Some condoms are inside coated with a topical anesthetic, such as xylocaine or benzocaine, to be interfaced against the male penis when unrolled and deployed, to desensitize the sensory nerve fibers of the male penis. This is to prevent premature ejaculation. Lubricated condoms contain a friction-reducing lubricant to ease initial vaginal penetration and vaginal intercourse. The lubricants used with condoms are FDA classified as medical devices and thoroughly tested to minimize irritation of both males and females, as well as to insure condom integrity.

A female sexual stimulant lubricant, such as a combination of menthol/L-arginine, described in U.S. Pat. Nos. 6,322,493 and 6,703,733, incorporated herein by reference, could be substituted for the current condom lubricants. In fact, the menthol/L-arginine topical preparation is classified by the FDA as a medical device, is FDA cleared for marketing, and published in the Federal Register under 510k number K021125. Pivotal to the FDA approval of the topical menthol/L-arginine sexual stimulant lubricant was a condom compatibility analysis, to insure that the menthol/L-arginine sexual stimulant lubricant did not cause, or contribute to, the weakening or breaking of a condom during use. The concern with using menthol/L-arginine sexual stimulant lubricant as a condom lubricant would be the indirect application of a topical menthol/L-arginine combination to the female clitoris. Pat. Nos. 6,322,493 and 6,702,733 discuss the direct application of the menthol/L-arginine application to the mucous membrane of the clitoris. The menthol component is for initial vasodilatation, reflex natural vaginal transudate lubrication, and to function as a lipophilic vehicle to transfer the L-arginine into the corpus cavernosa. The L-arginine, once within the stroma of the corpus cavemosa of the clitoris, is utilized to induce the nitric oxide synthase pathway to produce nitric oxide, cyclic GMP, smooth muscle relaxation, and a clitoral erection.

### FEMALE GENITAL ANATOMY

The vaginal mucosa is completely devoid of any sensory nerve endings. This is why a woman can use tampons and not really feel that the tampon is present. The vulvae has an abundance of sensory nerve endings, especially the introitis, the labia minora, and the clitoris. The clitoris, as the female analog of the male penis, has the greatest number of sensory nerve endings. Rationally, why introduce the menthol/L-arginine combination directly into the vagina as a lubricant, where, because of the lack of sensory nerve fibers and lack of a corpus cavemosa, the menthol/L-arginine would be expected to have little effect in sexual stimulation? Why not design a method to utilize a condom to directly deliver the menthol/L-arginine preparation directly to the entire vulvae, and especially to the clitoris?

### DESCRIPTION OF THE PRESENT INVENTION

The present invention relates to a method to deliver a topical sexual stimulant directly to a female's vulvar tissues, and especially the clitoris, by positioning the sexual enhancing lubricant on the proximal aspect of a completely unrolled and deployed condom. This would be accomplished by placing a band of topical sexual stimulant, like the menthol/L-arginine combination, on the proximal end of the condom, circumferentially, before the condom is rolled up for packaging. This would allow the sexual stimulant to remain sequestered within the initial several rolls of the condom, until the condom was deployed onto an erect penis. Once deployed and unrolled, and upon complete vaginal penetration, the topical sexual stimulant would be deposited not inside of the vagina, but directly onto the vulvae, and specifically the clitoris. Friction reducing, non-sexual stimulant lubricants, could be utilized in conjunction with the sequestered sexual stimulant lubricant, by adding the lubricant to the rolled up, condom upon final packaging, or to a fully deployed condom. With or without friction reducing lubricants, the sexual stimulating lubricant, positioned at the proximal end of the completely unrolled condom would insure the proper placement of the sexual stimulating menthol/L-arginine lubricant directly onto the vulvae and the clitoris during the repetitive actions of intercourse. In addition, the actions of active intercourse would have the net effect of rubbing the menthol/L-arginine into the clitoris tissues.

The sexual stimulating lubricant will be placed circumferentially at the proximal end of the condom, creating a band of lubricant. Application of the sexual stimulant may be accomplished through dipping, rolling, or placing a preformed band of lubricant on the proximal end. Placement of a preformed band may be accomplished through alteration of the lubricant state, liquid to solid. The lubricant may be altered to form a band by placing the sexual stimulant into a mold. When in this mold, the lubricant may be frozen or dehydrated. Once the stimulating band is placed on the condom, through dipping, rolling, or using a preformed band, the condom will be rolled toward the distal end, locking the sexual stimulant at the proximal end where it is most effective.

A further embodiment of the placement of the band of stimulant/lubricant may be accomplished by chilling the condom after it has been formed on an elongated mold. Such a mold form may be completely chilled or only its lower proximal end portion chilled through an internal chill line or by an external ducting of chilled fluid thereon. Such chilling would occur after a band of lubricant or stimulant were applied to the condom. Upon chilling and the slight solidification of such lubricant or stimulant, the condom may be rolled up for receipt within a sealable package.

A yet further embodiment of the present invention contemplates the application of a band of lubricant or stimulant at least partway along a portion of the condom which would be aligned with the dorsal aspect of the penis. The condom may be marked along such corresponding dorsal aspect thereof, by differing or distinguished surface characteristics and/or by marking the package of the condom to indicate preferred unrolling thereof prior to applying it to an erect penis.

The invention thus comprises a method of improving the use of a condom for delivery of a stimulant or lubricant treatment product to a female, comprising: forming a condom on a mold, the condom having a closed distal end and an open proximal end; and applying the treatment product on an arcuate band portion of the proximal end of the condom subsequent to the forming of the condom on the mold. The treatment product comprises a compound of menthol and L-arginine. The method may include one or more of the steps of: chilling the treatment product during the applying of the treatment product on the condom. The treatment product may be applied to a longitudinal portion of the condom. The longitudinal portion may comprise a narrow, elongated strip. The longitudinal portion may have indicia thereon to facilitate aligned use of the condom with the dorsal aspect of a male user of the condom. The condom may be rolled up and placed in a sealable package, The package may have a visible and tactile indicia thereon to indicate alignment of the treated longitudinal portion of the condom within the package. The mold may have an internal chilling means therein at a proximal portion therein. The mold may be chilled along a longitudinal portion thereof.

The invention may also comprise a condom for the delivery of a stimulant or lubricant to a female directly onto the vulvae and the clitoris during the repetitive actions of intercourse, the condom having a distal end and a proximal end, the condom comprising a band of a treatment compound of menthol and L-arginine around an annular portion of the proximal end of the condom. The condom may include an elongated band of the treatment compound along a longitudinal portion of said condom corresponding to the dorsal aspect of a user of the condom.

### BRIEF DESCRIPTION OF THE DRAWINGS

The objects and advantages of the present invention will become more apparent when viewed in conjunction with the following drawings in which:

FIG. 1 is a perspective view of a condom showing a band of lubricant or stimulant arranged thereon;

FIG. 2 is a side elevational view of a condom arranged on a mold for forming thereof and for application of band(s) of lubricant or stimulant thereon;

FIG. 3 is a side elevation of the condom shown in FIG. 2, in a partially rolled-up configuration;

FIG. 4 is a representation of a prior art condom in a partially rolled-up orientation without any lubricant or stimulant thereon; and

FIG. 5 is a representation of a package containing a condom, with a tactile mark on the package for indication the portion of the condom therein best oriented to the dorsal aspect of the male user.

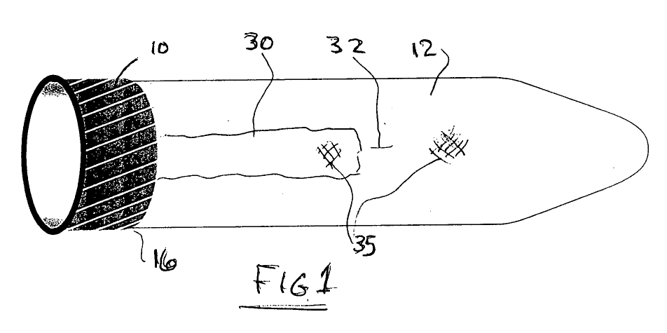

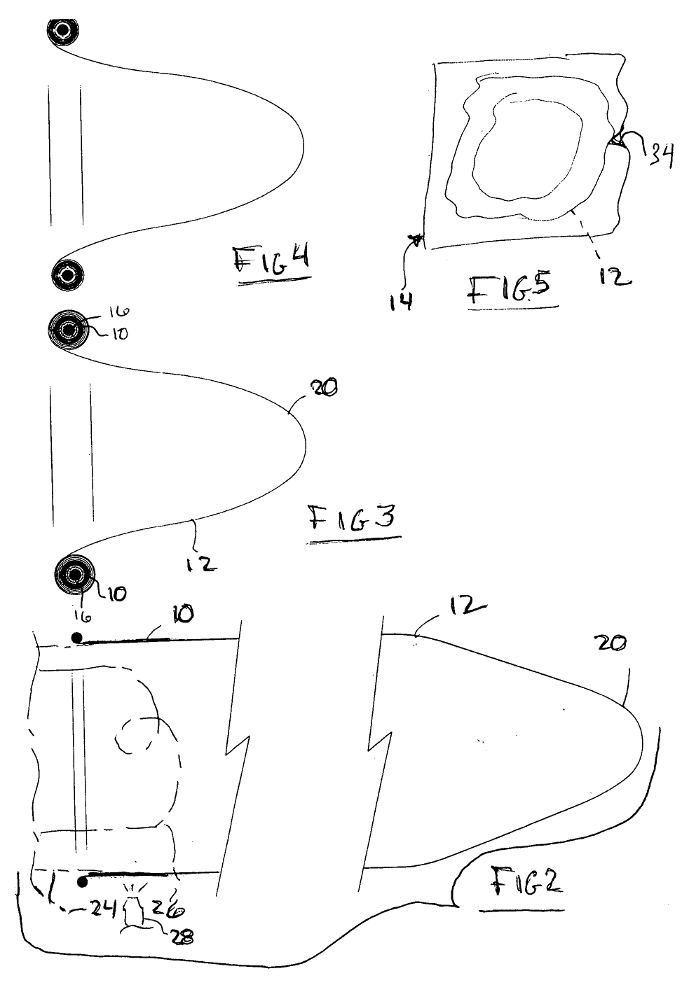

### DESCRIPTION OF THE PREFERRED EMBODIMENTS

Referring now to the drawings in detail, and particularly to FIG. 1, there is shown the present invention which comprises a sexual enhancing lubricant 10 on the proximal aspect of a completely unrolled and deployed condom 12. This would be accomplished by placing an annular band of topical sexual stimulant 10, preferably a menthol/L-arginine combination, on the proximal end 16 of the condom 12, before the condom 12 is rolled up for packaging, a package 14 being represented in FIG. 5. This would allow the lubricant and/or sexual stimulant 10 to remain sequestered within the initial several rolls of the condom 12, as represented in FIG. 3, until the condom 12 is deployed onto an erect penis. Once deployed and unrolled, and upon complete vaginal penetration, the topical sexual stimulant 10 would be deposited not inside of the vagina, but directly onto the vulvae, and specifically the clitoris. Friction reducing, non-sexual stimulant lubricants, could be utilized in conjunction with the sequestered sexual stimulant lubricant, by adding the lubricant to the rolled up, condom upon final packaging, or to a fully deployed condom. With or without friction reducing lubricants, the sexual stimulating lubricant, positioned at the proximal end 16 of the completely unrolled condom 12 would insure the proper placement of the sexual stimulating menthol/L-arginine lubricant directly onto the vulvae and the clitoris during the repetitive actions of intercourse. In addition, the actions of active intercourse would have the net effect of rubbing the menthol/L-arginine into the clitoris tissues.

The sexual stimulating lubricant will be placed circumferentially at the proximal end 16 of the condom 12, creating the annular band of lubricant 10. Application of the sexual stimulant may be accomplished through dipping, rolling, spraying or placing a preformed band of lubricant on the proximal end. Placement of a preformed band 10 may also be accomplished through alteration of the lubricant state, liquid to solid. The lubricant 10 may be altered to form a band by placing the sexual stimulant into a mold. When in this mold, the lubricant 10 itself may be frozen or dehydrated. Once the stimulating band is placed on the condom, through dipping, rolling, or using a preformed band, the condom will be rolled toward its distal end 20, as represented in FIG. 3, locking the sexual stimulant at the proximal end 16, where it is most effective.

A further embodiment relative to the application of the band of stimulant/lubricant 10 may be accomplished by chilling the condom after it has been formed on an elongated mold 22, as represented in FIG. 2. Such a mold form 22 may be completely chilled or only its proximal end portion 24 chilled through an internal chill line 26 or by an external ducting of chilled fluid thereon by a sprayer 28. Such chilling may preferably occur during or after a band of lubricant or stimulant 10 is being/has been applied to the condom 12. Upon chilling and the slight solidification of such lubricant or stimulant, the condom 12 may be rolled up, for subsequent receipt within the sealable package 14, as shown in FIG. 5.

A yet further embodiment of the present invention contemplates the application of an elongated, longitudinally directed band of lubricant or stimulant 30 at least partway along a longitudinal portion 32 of the condom 12, as also represented in FIG. 1, which longitudinal portion 30 which should, during use, be aligned with the dorsal aspect of the penis. The condom 12 may also be marked along such longitudinal portion 32, corresponding to and facilitating the alignment with the dorsal aspect of the user, by differing or distinguished surface characteristics 35 thereon, and/or by marking the package 14 by a visible and/or tactile notch 34 containing the condom to indicate preferred unrolling thereof prior to applying it to an erect penis.

We claim:

1. A method of improving the use of a condom for delivery of a stimulant or lubricant treatment product to a female, comprising:
forming a condom on a mold, said condom having a closed distal end and an open proximal end; and
applying said treatment product on an arcuate band portion of said proximal end of said condom subsequent to said forming of said condom on said mold.

2. The method as recited in claim 1, wherein said treatment product comprises a compound of menthol and L-arginine.

3. The method as recited in claim 1, including the step of:
chilling said treatment product during said applying of said treatment product on said condom.

4. The method as recited in claim 1, wherein said treatment product is applied to a longitudinal portion of said condom.

5. The method as recited in claim 4, wherein said longitudinal portion comprises a narrow, elongated strip.

6. The method as recited in claim 4, wherein said longitudinal portion has indicia thereon to facilitate aligned use of said condom with the dorsal aspect of a male user of said condom.

7. The method as recited in claim 4, wherein said condom is rolled up and placed in a sealable package, said package has a visible and tactile indicia thereon to indicate alignment of said longitudinal portion of said condom within said package.

8. The method as recited in claim 3, wherein said mold has an internal chilling means therein at a proximal portion therein.

9. The method as recited in claim 4, wherein said mold is chilled along a longitudinal portion thereof.

10. A condom for the delivery and application of a stimulant or lubricant to a female directly onto her vulvae and clitoris during the repetitive actions of intercourse, said condom having a distal end and a proximal end, said condom comprising:
a band of a treatment compound of menthol and L-arginine around an annular portion of said proximal end of said condom.

11. The condom as recited in claim 10, including an elongated band of said treatment compound along a longitudinal portion of said condom corresponding to the dorsal aspect of a user of said condom.
